(19) United States
(12) Patent Application Publication
(10) Pub. No.: US 2025/0009565 A1
(43) Pub. Date: Jan. 9, 2025

(54) A MEDICAL DRESSING

(71) Applicant: MÖLNLYCKE HEALTH CARE AB, GÖTEBORG (SE)

(72) Inventors: Conny Jakobsson, Lerum (SE); Malin Mårtensson, Borås (SE)

(21) Appl. No.: 18/711,672

(22) PCT Filed: Nov. 26, 2021
(86) PCT No.: PCT/EP2021/083132

§ 371 Date: May 20, 2024

(51) Int. Cl. A61F 13/06; A61F 13/02

(52) U.S. Cl. CPC A61F 13/064; A61F 13/023; A61F 13/0266

(57) ABSTRACT

Described is a medical dressing for application onto a body part, wherein the medical dressing has a longitudinal (y) extension and a lateral (x) extension and is symmetric about a longitudinal (y) center line and asymmetric about a lateral (x) center line. The dressing has at least a first, centrally disposed release liner comprising at least one graphical icon indicating a correct application of the medical dressing onto the body part. Also described is a method for manufacturing the medical dressing, to a sterile package that includes the medical dressing and to a multi-pack that includes a plurality of the sterile packages.

### TECHNICAL FIELD

The present disclosure generally relates to a medical dressing for application onto a body part, preferably the heel, wherein the dressing has a longitudinal (y) extension and a lateral (x) extension and is symmetric about a longitudinal (y) center line and asymmetric about a lateral (x) center line. The dressing comprises at least a first, centrally disposed release liner comprising at least one graphical icon indicating a correct application of the medical dressing onto the body part. The present disclosure also relates to a method for manufacturing such a medical dressing, to a sterile package comprising the medical dressing and to a multi-pack comprising a plurality of such sterile packages.

### BACKGROUND

Adhesive medical dressings are frequently used in wound care, both for the purpose of treating wounds and scars and for the purpose of preventing these from occurring in the first place. An adhesive medical dressing typically comprises an adhesive wound contact layer arranged to contact the skin or wound of a patient, a wound pad, and a top layer, often referred to as a backing layer. To protect the dressing from contamination, a release liner may be applied to the adhesive wound contact layer.

For certain areas of the human body, the application of a dressing is more difficult than others. Such body areas may be referred to as “hard to dress” areas, and include convex body parts, e.g. the heels, the sacrum and the elbows.

The application of a medical dressing onto the heel of a patient is typically challenging. The heel comprises bony prominences where the skin is more vulnerable and susceptible to the formation of pressure ulcers. Examples of such bony prominences of the heel include i.a. under the heel, on the outside of the heel and the malleoli.

Accordingly, it is generally desired to cover such bony prominences to reduce the risk of pressure ulcer formation. The foot sole as well as the lateral and medial sides of the foot must generally be covered. Applying a dressing to another convex part of the human body, such as an elbow or sacrum may be challenging for the same reasons.

To date, dressings particularly adapted for convex body parts, such as heels exist on the market. These dressings may be categorized into cup-shaped dressings and “flat” adhesive dressings.

Cup-shaped dressings typically have a “fixed” design; i.e. it is not possible to adapt the “cup” to suit a certain convex body part or a certain patient. Cup-shaped dressing also come in one size which may not fit all convex body parts and not all patients since the anatomy differs from patient to patient.

Flat adhesive dressings particularly aimed for application onto convex body parts, such as the heels, solve some of the problems associated with cup-shaped dressings, since these dressings allow for more flexibility during application.

Flat adhesive heel dressings are typically large and have a rather “complex” design. For example, heel dressings are often asymmetric in shape and may comprise various protruding dressing parts. The protruding parts are adapted to protect the most vulnerable parts of the foot and heel of a patient.

One problem with flat adhesive heel dressings is that the dressings are difficult to apply to the patient correctly. In a care facility or hospital, the application of the dressing must be carried out by caregivers and medical staff.

During application, the dressing and its protruding parts may need to be folded to conform to the shape of the convex body part, and this typically results in various overlaps of the dressing portions. Consequently, there is a risk for forming gaps and/or folds when applying the medical dressing to e.g. the heel of a patient. Furthermore, an incorrect application may significantly impair the wear time of the dressing, especially if the skin needs to be detached and re-attached from the skin as is the case when the skin needs to be inspected for signs of pressure ulcer formation.

In addition, the direction by which the dressing should be applied to the heel or another convex body part may be difficult to understand. Many times, the dressing is applied “upside down”. Such initial misplacement of the dressing may result in a poor coverage of the areas aimed for protection.

In view of this it is desired to prevent pressure ulcers from forming on convex body parts, particularly the heels of a patient, and to provide a means to facilitate the application of a dressing adapted for convex body parts. Such means should desirably relieve the burden for medical staff in a care facility or hospital dealing with pressure ulcers and dressings being difficult to apply correctly onto a convex body part, such as the heels.

### SUMMARY

In view of the above-mentioned problems, it is an object of the present disclosure to provide improvements in the handling and application of dressings suited for convex body parts, and to provide a means to facilitate the situation for medical staff in such situations.

According to a first aspect, there is provided a medical dressing for application onto a body part, preferably the heel, wherein the dressing has a longitudinal (y) extension and a lateral (x) extension and is symmetric about a longitudinal (y) center line and asymmetric about a lateral (x) center line; the dressing comprising a backing layer, an adhesive skin contact layer and a pad arranged between the backing layer and the adhesive skin contact layer, wherein the dressing comprises a release liner assembly being co-extensive with the adhesive skin contact layer, wherein the release liner assembly comprises a centrally disposed first release liner covering from 50 to 90% of the surface area of the adhesive skin contact layer, and a plurality of edge release liners, wherein the first release liner comprises at least one graphical icon indicating a correct application of the medical dressing onto the body part.

The present disclosure is based on the realization that the graphical icon(s) provided on the first, larger release liner offers a direct and clear guidance for the medical staff in how to correctly apply the dressing onto a body part, e.g. the heel. While instructions relating to a correct use and application of the dressing may be provided in an instruction manual (“Instructions for use”, IFU), inserted into the multi-pack in which the individual dressings are stored, such IFUs are generally not used or present in the actual care situation. By means of the present disclosure, the medical staff is offered a direct, “on site” and “in situation” assistance such that the dressing can be properly applied onto the body part of the patient. Furthermore, the risk of applying the dressing incorrectly, such that e.g. gaps and folds are formed, is significantly reduced. The risk for developing pressure ulcers is thereby considerably reduced.

The dressing is symmetrical about a longitudinal center line and asymmetrical about a lateral center line. Accordingly, the dressing has a shape that makes it difficult to determine in what direction the dressing should be applied onto the body part, e.g. the heel. A well-recognized problem with such dressings is that the dressings are applied upside down. Accordingly, such initial misplacement of the dressing may result in that the most vulnerable areas are not covered properly.

The first release liner onto which the at least one graphical icon is provided has a large size. Accordingly, the provision of the graphical icon(s) onto the first release liner, e.g. by means of printing techniques, is facilitated, and the graphical icon(s) is/are visibly recognizable on the first release liner.

The medical staff may, after removal of the first release liner, utilize the first release liner as an “on site” support in the process of applying the remaining parts of the dressing onto the body part. The body part is typically a convex body part, e.g. the heel.

Preferably, the backing layer and the adhesive skin contact layer extend beyond the periphery of the pad to form a border portion around the pad.

Accordingly, an improved adhesion to the skin is achieved, and the wear time is enhanced.

In exemplary embodiments, the medical dressing comprises a plurality of protruding portions, and wherein the edge release liners are arranged to cover at least parts of the protruding portions.

The protruding portions may be defined by parts of the pad and the border portion of the dressing. Typically, the protruding portions are defined by parts of the border portion of the dressing. The protruding portions may be adapted to be folded and applied partially over another part of the medical dressing during application onto a convex body part, such as the heel.

In the process of applying the medical dressing to the convex body part, e.g. the heel, the first release liner is first removed. Subsequently, the dressing part uncovered by the first release liner is positioned onto the convex body part, e.g. a heel. During this step, the edge release liners may remain adhered to the adhesive skin contact layer. Thereafter, the remaining (protruding) portions of the dressing are carefully anchored onto the remaining parts of the convex body part while simultaneously removing the edge release liners in consecutive order. In the application of the dressing onto the convex body part, some areas of the dressing may need to be folded over the remaining parts of the dressing such that the various dressing portions overlap. The graphical icon(s) provided on the first release liner guide the staff in how to properly fold and apply the protruding portions of the dressing.

In exemplary embodiments, the at least one graphical icon is disposed in an area of the first release liner that overlaps with the pad.

Preferably, a relatively large area of the pad is “covered” by the first release liner. The pad and the first release liner are centrally disposed on the dressing. In embodiments where the first release liner is transparent, the underlying pad secures that the at least one graphical icon is clearly recognizable to a viewer.

In embodiments, the first centrally disposed release liner is symmetric about the longitudinal (y) center line and has a longitudinal extension corresponding to the longitudinal extension of the adhesive skin contact layer in the area where the first release liner is arranged, and a maximum lateral (x) extension corresponding to from 50-90%, preferably from 55 to 75% of the maximum lateral (x) extension of the adhesive skin contact layer.

Accordingly, the first release liner is co-extensive with the adhesive skin contact layer in the longitudinal direction.

The first release liner is configured to be removed first from the adhesive skin contact layer.

Accordingly, the first release liner may be utilized as an “on site” support that the staff can utilize in the subsequent steps of the application of the dressing onto the convex body part.

In exemplary embodiments, the first release liner portion comprises at least a first set of graphical icons, wherein each graphical icon within the first set of icons comprises a pictorial representation of a step associated with the correct application of the medical dressing onto the body part, preferably the heel.

The pictorial representations illustrate the steps in the application procedure in consecutive order.

The step(s) associated with the correct application of the medical dressing may be selected from: a correct removal and/or order of removal of the release liner assembly, a correct positioning of the medical dressing onto the heel, a correct application of the dressing onto the heel, and a medical dressing applied correctly onto the heel.

The first set of graphical icons typically represents a series comprising the steps of a) first removing the first release liner from the dressing, b) correctly positioning of the medical dressing onto the heel, c) correctly applying the portions of the dressing onto the heel (which, in embodiments, may also illustrate the order by which the edge release liners should be removed from the adhesive skin contact layer), and d) an illustration of a correctly applied medical dressing onto the heel. Preferably, a correct application onto the malleoli is visualized.

In exemplary embodiments, the at least one graphical icon within the first set of graphical icons comprises a pictorial representation indicating the correct positioning of the medical dressing onto the heel, and wherein the pictorial representation comprises a guiding line indicating the correct positioning of the medical dressing onto the heel.

After the first release liner has been removed, the dressing is to be positioned onto the skin of a patient. Typically, such positioning is made while the edge release liners remain adhered to the adhesive skin contact layer. The positioning of the dressing onto the heel is a critical step since this step is often carried out incorrectly. The asymmetric character of the dressing, and of the pad, may result in positioning the dressing upside down. Such initial misplacement will result in the remaining parts of the dressing being incorrectly applied to the convex body part. Accordingly, the areas of the convex body part in most need of protection may not be covered.

In embodiments, the first set of graphical icons comprises at least four graphical icons, and wherein the graphical icons of the first set of graphical icons are arranged in at least a first row and a second row on the first release liner.

The distance, d1 between a first graphical icon of the first row and a neighboring graphical icon within the first row is the same as the distance, d2, between the first graphical icon within the first row and a neighboring graphical icon within the second row.

Accordingly, the distance between a first graphical icon and a second graphical icon is the same in the lateral and longitudinal directions. The graphical icons within a first set of graphical icons are disposed such that the order of the steps can be followed in a clear and communicative manner.

In embodiments, the first release liner and/or the edge release liners further comprise(s) at least one printed texted word and/or a graphical branding, wherein the orientation of the printed texted word and/or graphical branding, indicates the orientation by which the dressing should be positioned onto the convex body part.

Typically, the at least one texted word and/or graphical branding is readable in the direction by which the dressing is to be positioned onto the body part.

This is to further emphasize the correct positioning of the dressing and to prevent misplacement of the dressing onto the (convex) body part.

The graphical branding may be a trademark, a logotype or a symbol.

Typically, at least the first release liner of the release liner assembly is transparent or white.

This is beneficial to provide a visually distinct difference between the graphical icons and the release liner.

In exemplary embodiments, the first set of graphical icons are disposed on the first release liner such that at least 50%, preferably at least 70% of the graphical icons of the first set of graphical icons overlap with the the pad.

This is particularly advantageous in embodiments where the release liner is transparent. The underlying pad, which is typically white in color, then provides a distinct contrast to the graphical icons provided on the first release liner.

In embodiments, at least the first release liner of the release liner assembly comprises a polymeric film selected from a polyethylene film, a polypropylene film and a polyurethane film.

A polymeric film allows for printing, welding, compressing and/or embossing the graphical icons, and the graphical branding(s), if present, onto the first release liner.

Typically, the at least one graphical icon is a printed graphical icon formed by a colored ink.

The graphical icons are typically printed on the first release liner. Printing allows for a facilitated application of the graphical icons onto the first release liner. The colored ink may have a hydrophobic character to prevent the ink from dissolving in contact with body fluids.

In embodiments, at least the first release liner of the release liner assembly has a surface tension that is higher than the surface tension of the colored ink.

This allows for an improved adherence of the colored ink to the first release liner.

In embodiments, a central area of the first release liner is void of printed graphical icon(s) and texted word(s) or graphical branding(s), if present, wherein the central area is an area circumventing the longitudinal (y) center line and having a lateral (x) extension of from 5 to 20%, preferably from 5 to 15% of the maximum lateral (x) extension of the first release liner.

The first release liner is preferably void of any printed graphical icon(s), texted words or graphical branding(s) along the longitudinal center line and a small area circumventing the longitudinal center line.

When the dressing is packaged and positioned in a sterile pack, a variety of control measures must be performed. Typically, such control measures are performed by an automated process and control device which illuminates the dressing inside the sterile package and measures a variety of parameters of the dressing to secure that the dressing is correctly packaged and positioned. One such parameter is the length of the dressing along the longitudinal center line. If printed markings, e.g. in the form of graphical icon(s) or graphical branding(s) are present in vicinity of the edges of the pad, such printed markings may be mistaken as pad edges, which impairs the packaging control performed by the control system. Accordingly, the first release liner is preferably void of printed markings along the longitudinal center line and in the area circumventing the longitudinal center line.

In embodiments, the colored ink reflects light in the range of from 430 to 500 nm.

If printed graphical icon(s) and/or graphical branding(s) are provided on the first release liner (and in vicinity of the pad edges), it may be beneficial to utilize a colored ink that reflects light in the range of from 430 to 500 nm. This way, the printed markings (graphical icons and brandings) are experienced as transparent by the automated control system utilized during packaging of the dressing into a sterile package. Accordingly, the dressing can be correctly packaged without risking interfering with the automated control device.

According to another aspect, there is provided a method for manufacturing a medical dressing comprising:
a) providing a medical dressing having a longitudinal (y) extension and a lateral (x) extension; the medical dressing being symmetric about a longitudinal (y) center line and asymmetric about a lateral (x) center line; the dressing comprising a plurality of protruding portions, wherein the dressing comprises a backing layer, an adhesive skin contact layer and a pad arranged between the backing layer and the adhesive skin contact layer, wherein the backing layer and the adhesive skin contact layer extend beyond the periphery of the pad to form a border portion around the pad;
b) providing a release liner assembly comprising a first release liner and a plurality of edge release liners;
c) printing at least one graphical icon onto the first release liner, wherein the graphical icon indicates a correct application of the medical dressing onto a body part, e.g. the heel;
d) applying the plurality of edge release liners onto the adhesive skin contact layer of at least parts of the protruding portions of the medical dressing,
e) applying the first release liner onto a central portion of the adhesive skin contact layer such that the first release liner covers from 50 to 90% of the surface area of the adhesive skin contact layer, and
f) trimming the first release liner and the edge release liners of the release liner assembly such that a medical dressing comprising a release liner assembly being co-extensive with the adhesive skin contact layer is formed.

According to another aspect, there is provided a sterile package comprising the medical dressing as described hereinbefore, wherein the sterile package comprises a second set of graphical icons, wherein each graphical icon within the second set of icons comprises a pictorial representation of a step associated with the correct application of the medical dressing, and wherein at least one graphical icon within the first set of graphical icons of the first release liner corresponds to at least one graphical icon within the second set of graphical icons.

The second set of graphical icons may comprise a more comprehensive set of graphical icons, comprising additional graphical icons with pictorial representations of additional steps in the application procedure. If the medical staff needs additional guidance, he/she may turn to the sterile package for a more comprehensive guidance.

Typically, at least two, e.g. at least three, e.g. at least four of the graphical icons of the first set of graphical icons correspond to the graphical icons of the second set of graphical icons.

According to yet another aspect, there is provided a multi-pack comprising a plurality of sterile packages as described hereinbefore, wherein the multi-pack comprises a third set of graphical icons, wherein each graphical icon within the third set of icons comprises a pictorial representation of a step associated with the correct application of the medical dressing, wherein at least one of the graphical icons within the third set of graphical icons corresponds to at least one of the graphical icons within the first and the second sets of graphical icons.

Further features of, and advantages with, the present disclosure will become apparent when studying the appended claims and the following description. The skilled addressee realizes that different features of the present disclosure may be combined to create embodiments other than those described in the following, without departing from the scope of the present disclosure.

### BRIEF DESCRIPTION OF THE DRAWINGS

The various aspects of the present disclosure, including its particular features and advantages, will be readily understood from the following detailed description and the accompanying drawings, in which:

FIG. 1a illustrates a medical dressing according to an exemplary embodiment of the present disclosure, seen from the release liner assembly.

FIG. 1b is a split view of the dressing in FIG. 1a.

FIG. 2 illustrates a first release liner comprising a first set of graphical icons according to an exemplary embodiment of the present disclosure.

FIG. 3 illustrates two alternative heel dressings in accordance with exemplary embodiments of the present disclosure.

FIG. 4a illustrates a sterile package comprising a medical dressing according to an exemplary embodiment of the present disclosure.

FIG. 4b illustrates a multi-pack comprising a plurality of sterile packages according to an exemplary embodiment of the present disclosure.

### DETAILED DESCRIPTION

The present disclosure will now be described more fully hereinafter with reference to the accompanying drawings, in which currently preferred embodiments of the present disclosure are shown. The present disclosure may, however, be embodied in many different forms and should not be construed as limited to the embodiments set forth herein; rather, these embodiments are provided for thoroughness and completeness, and fully convey the scope of the present disclosure to the skilled person. Like reference characters refer to like elements throughout.

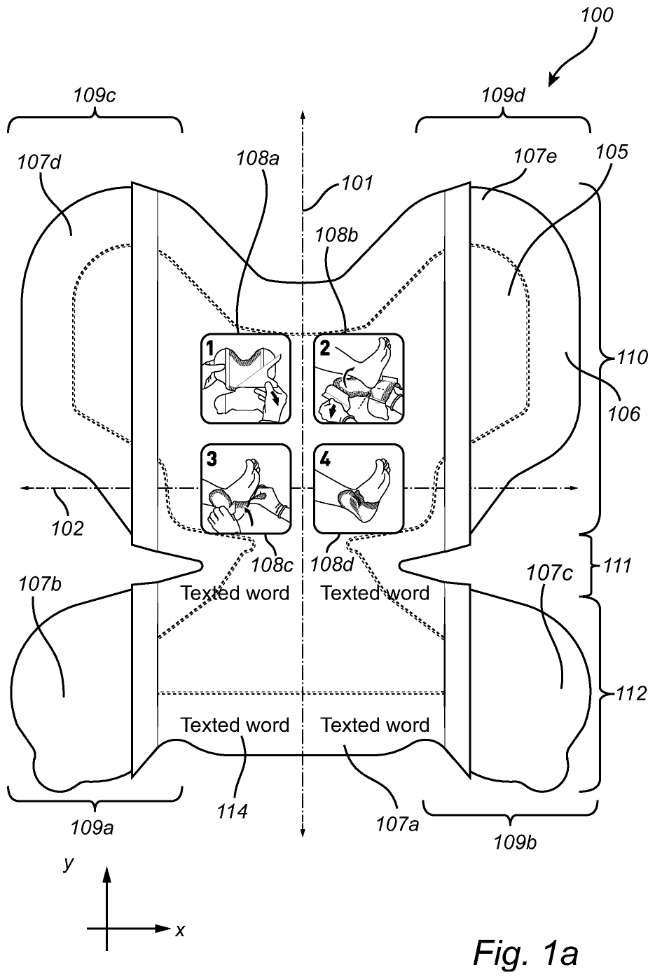

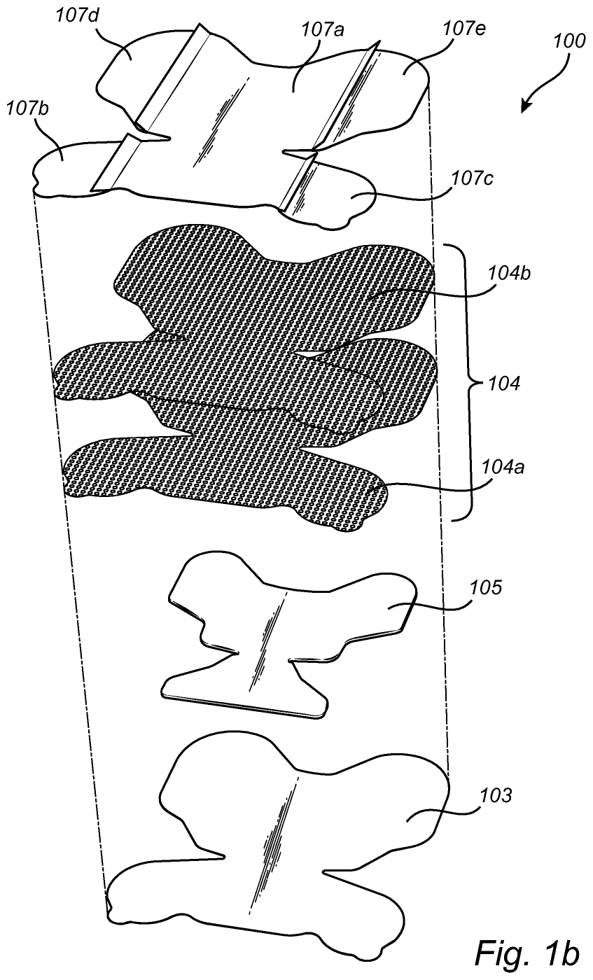

With reference to FIGS. 1a and 1b, a medical dressing 100 for application onto a body part, preferably the heel according to the present disclosure is conceptually illustrated.

The medical dressing (100) has a longitudinal (y) extension and a lateral (x) extension and is symmetric about a longitudinal (y) center line 101 and asymmetric about a lateral (x) center line 102; the medical dressing comprising a backing layer 103, an adhesive skin contact layer 104 and a pad 105 arranged between the backing layer 103 and the adhesive skin contact layer 104, wherein the dressing comprises a release liner assembly 107a-e being co-extensive with the adhesive skin contact layer 104, wherein the release liner assembly 107a-e comprises a centrally disposed first release liner 107a covering from 50 to 90% of the surface area of the adhesive skin contact layer 104, and a plurality of edge release liners 107b-e, wherein the first release liner 107a comprises at least one graphical icon 108a-d indicating a correct application of the medical dressing 100 onto the body part, e.g. the heel.

As used herein, the term “graphical icon” means a pictorial representation, a graphical figure, image, sign or visual element that aids in communicating information and instructions for use and application of the dressing to the caregiver. The at least one graphical icon is typically printed on the first release liner. Accordingly, the graphical icon(s) is formed by a print pattern. The graphical icon(s) may also be formed by, or complemented with, a weld pattern, a compressed pattern, an embossed pattern, a material deposition pattern, or combinations thereof. The graphical icon may be associated with words or numbers to convey the information in a consumer-friendly manner, e.g. to indicate the order of interpretation of the graphical icons.

The release liner assembly 107a-e is co-extensive with the adhesive skin contact layer 104. Accordingly, the surface area of the release liner assembly 107a-e may correspond to the surface area of the adhesive skin contact layer 104.

The release liner assembly 107a-e comprises a centrally disposed, first release liner, and a plurality of edge release liners.

The term “centrally disposed” means that the first release liner is arranged along the longitudinal center line. The first release liner is symmetric about the longitudinal center line. The longitudinal extension of the first release liner corresponds to the longitudinal extension of the adhesive skin contact layer. The maximum lateral (x) extension of the first release liner corresponds to from 50 to 90%, preferably from 55 to 75% of the maximum lateral extension of the adhesive skin contact layer.

The term “plurality of edge release liners” means at least two edge release liners, e.g. at least three edge release liners, e.g. at least four edge release liners. The edge release liners cover the peripheral edges of the adhesive skin contact layer not covered by the first release liner. The peripheral edges of the adhesive skin contact layer (and the dressing) may represent protruding portions.

A “body part” may be any part of the human body. Typically, the body part is a “convex body part”. The convex body part may be the heel, the sacrum or the elbow. In the context of the present disclosure, the heel is particularly envisaged.

In embodiments, the medical dressing of the present disclosure is a heel dressing.

The medical dressing is asymmetric about a lateral (x) center line 102. The pad 105 is typically also asymmetric about the lateral (x) center line 102. The asymmetric construction of the dressing typically results in misplacement of the dressing onto the convex body part. In many cases, the dressing is placed upside down. Furthermore, if the dressing comprises a plurality of protruding portions that are intended to be wrapped or folded over another part of the dressing, the misplacement and wrapping of such protruding portion may result in poor coverage of the most vulnerable areas of the convex body part, as well as detachment of the dressing from the skin, which reduces the wear time of the dressing.

The graphical icon(s) 108a-d provided on the first release liner 107a secures proper placement and proper application of the remaining parts of the dressing onto the convex body part.

Typically, the backing layer 103 and the adhesive skin contact layer 104 extend beyond the periphery of the pad 105 to form a border portion 106 around the pad 105.

As can be seen in FIG. 1a, the medical dressing 100 comprises a plurality of protruding portions 109a-d, and wherein the edge release liners 107b-e are arranged to cover at least parts of the protruding portions 109a-d.

As used herein, the term “protruding portion” means a portion of the dressing extending beyond a central area of the dressing. The protruding portion may extend obliquely from the longitudinal center line. The protruding portion typically has a lateral and a longitudinal extension being less than 50% of the respective maximum lateral and longitudinal extension of the dressing.

In the exemplary embodiment illustrated in FIG. 1a, the medical dressing 100 comprises a first portion 110, a second portion 111, and a third portion 112, wherein the second portion 111 is arranged between and adjoining the first 110 and the third 112 portion.

The width of the second portion 111 is smaller than the width of the first, and the second portion, respectively.

For example, the width of the first portion 110 may be in the range of from 15 to 30 cm, e.g. from 20 to 25 cm. The width of the second portion 111 may be in the range of from 5 to 15 cm, e.g. from 7 to 12 cm. The width of the third portion 112 may generally correspond to the width of the first portion 110.

The width is measured between the peripheral edges of the dressing in the lateral direction of the dressing.

The first portion 110 may have a longitudinal extension corresponding to from 45 to 60% of the maximum longitudinal extension of the medical dressing.

The second portion 111 may have a longitudinal extension corresponding to from 5 to 15% of the maximum longitudinal extension of the medical dressing.

The third portion 112 may have a longitudinal extension corresponding to from 25 to 40% of the maximum longitudinal extension of the medical dressing.

In embodiments, the length of the first portion 110 is from 10 to 15 cm. The length of the second portion 111 may be from 1 to 3 cm. The length of the third portion 112 may be from 5 to 10 cm.

The length is measured in the longitudinal direction of the dressing.

In the embodiment illustrated in FIG. 1a, the second portion 111 is typically the portion onto which the heel should initially be positioned. The first portion 110 is the portion adapted to cover the heel, ancle and malleoli. The third portion 112 is adapted to be folded partially over the second portion 112 to enhance the coverage of the malleoli and parts of the foot sole.

In FIG. 1a, the first portion comprises two protruding portions 109c-d extending obliquely from the smaller second portion 111 and from the longitudinal center line 101. Furthermore, the third portion 112 comprises two protruding portions 109a-b extending obliquely from the second portion 111.

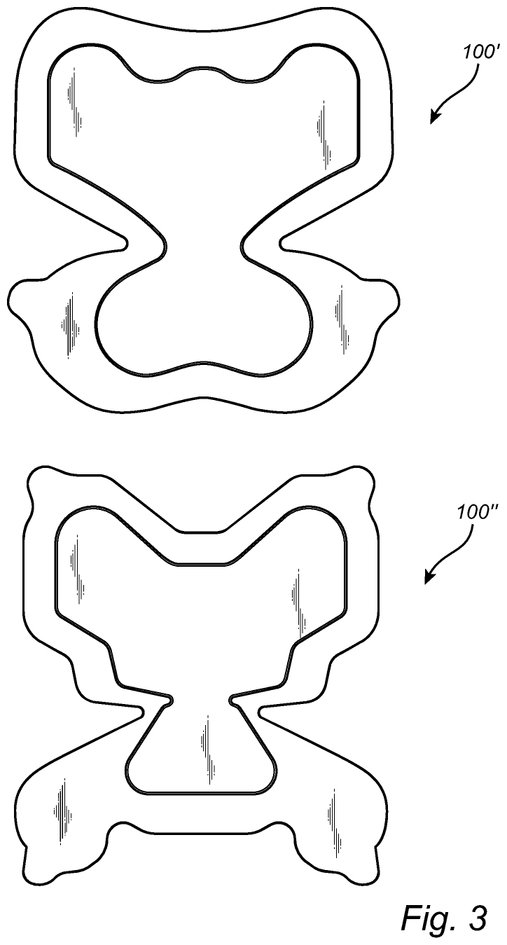

In this regard, it must be noted that the dressing illustrated in FIG. 1 represents one example of a dressing of the present disclosure. The present disclosure is by no means limited to the dressing shape illustrated in FIG. 1. For example, FIG. 3 represents alternative shapes of the medical dressing of the present disclosure.

The first release liner 107a has a large size and is configured to cover from 50 to 90% of the surface area of the adhesive skin contact layer 104. The large size of the first release liner enables provision, e.g. by printing techniques, of the graphical icon(s) 108a-d such that these are clearly recognizable on the medical dressing.

Typically, at least one graphical icon 108a-d is disposed in an area of the first release liner 107a that overlaps with the pad 105.

As used herein, the term “overlap” means that the first release liner overlies the pad but need not necessarily be in direct contact with the pad.

As illustrated in FIG. 1a, the first centrally disposed release liner 107a is symmetric about the longitudinal (y) center line 101 and has a longitudinal extension corresponding to the longitudinal extension of the adhesive skin contact layer 104 in the area where the first release liner 107a is arranged, and a maximum lateral (x) extension corresponding to from 50-90%, preferably from 55 to 75% of the maximum lateral (x) extension of the adhesive skin contact layer 104.

This configuration is beneficial to prevent potential contaminants from entering the dressing. Furthermore, the large size of the first release liner 107a facilitates printing of a plurality of graphical icons thereto. Also, instead of turning to an instruction manual for use (IFU) stored in the multi-pack of the dressing, the caregiver may utilize the large first release liner 107a as an on-site support in the actual situation of applying the dressing onto the convex body part.

The first release liner 107a is configured to be removed first from the adhesive skin contact layer 104.

As mentioned hereinbefore, after removal, the first release liner 107 provided with at least one graphical icon 108a-d may be utilized as a support for the medical staff in the subsequent application of the dressing onto the convex body part, and the subsequent removal of the remaining edge release liner portions of the release liner assembly 107a-e. Accordingly, an “on site” and “in situation” guidance to facilitate the application of the dressing is provided.

The first release liner 107a overlaps and extends slightly beyond the edge release liners 107b-d. The edge release liner portions 107b-d are typically folded to enable the caregiver to grasp the edge release liner portions 107b-d and remove these from the adhesive surface of the dressing.

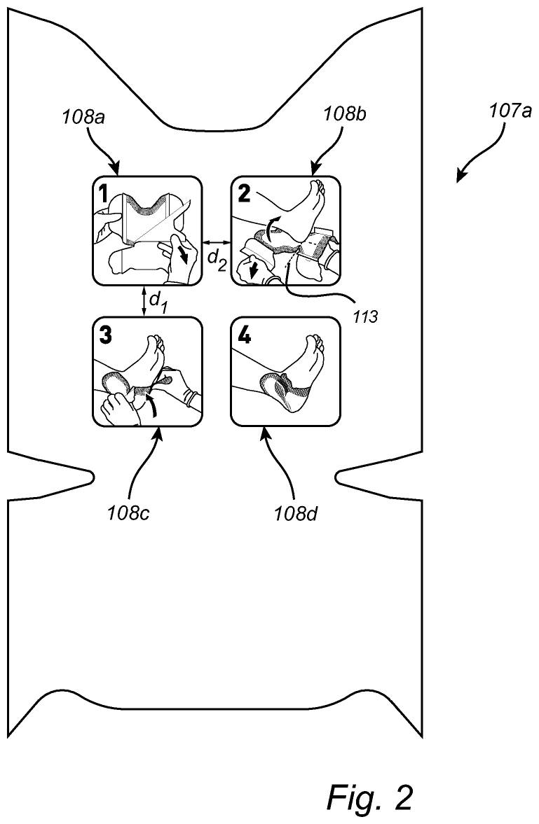

As illustrated in FIGS. 1a and 2, the first release liner 107a comprises at least a first set of graphical icons 108a-d, wherein each graphical icon within the first set of graphical icons 108a-d comprises a pictorial representation of a step associated with the correct application of the medical dressing 100 onto the body part.

As used herein, the term “first set of graphical icons” means at least two graphical icons, e.g. at least three graphical icons, preferably at least four graphical icons.

As used herein, the term “pictorial representation” means an image or a figure illustrating a step in the application of the dressing onto the body part. The step indicated by the pictorial representation may be selected from: a correct removal and/or order of removal of the release liner assembly, a correct positioning of the medical dressing onto the heel of a patient, a correct application of the medical dressing onto the heel of a patient, and a medical dressing applied correctly onto the heel of a patient. One important step of the pictorial representation is a correct application of the dressing onto the malleoli.

The pictorial representations are consumer-friendly, simple, and clear and convey the correct application of the dressing onto the (convex) body part.

As best illustrated in FIG. 2, at least one graphical icon within the first set of graphical icons 108a-d comprises a pictorial representation indicating the correct positioning of the medical dressing 100 onto the heel, and wherein the pictorial representation comprises a guiding line 113 indicating the correct positioning of the medical dressing 100 onto the heel (see 113 and 108b in FIGS. 1a and 2).

The provision of the guiding line 113 prevents misplacement of the dressing onto the heel. Instead, a correct initial placement of the dressing onto the heel is achieved, which also prevents the dressing from being positioned upside down.

As used herein, the term “guiding line” means at least one printed line indicating the position on the dressing where the heel should initially be placed. The printed line(s) may be continuous, bolded, discontinuous or dotted line(s).

In FIGS. 1a and 2, the first set of graphical icons 108a-d comprises at least four graphical icons, and wherein the graphical icons of the first set of graphical icons 108a-d are arranged in at least a first row and a second row on the first release liner 107a.

Accordingly, four consecutive steps are illustrated to guide the medical staff throughout the procedure of applying the dressing onto the convex body part.

The first graphical icon 108a may comprise a pictorial representation illustrating how to remove the first release liner from the adhesive skin contact layer.

The second graphical icon 108b may comprise a pictorial representation illustrating where and how to position the medical dressing onto the heel. The pictorial representation comprises a guiding line 113 indicating where the heel should be positioned on the adhesive skin contact layer of the dressing.

The third graphical icon 108c may comprise a pictorial representation illustrating the application of the dressing onto the malleoli and the ankle. More particularly, the pictorial representation of the third graphical icon illustrates how the protruding portions of the dressing should be applied to protect the malleoli.

The fourth graphical icon 108d may comprise a pictorial representation illustrating the heel dressing when applied to the heel and part of the foot of the patient.

The first set of graphical icons 108a-d may comprise additional graphical icons indicating additional steps of the application procedure. The first release liner 107a may comprise more than one first set of graphical icons 108a-d.

The distance, d1 between a first graphical icon 108a of the first row and a neighboring graphical icon 108b within the first row is generally the same as the distance, d2, between the first graphical icon 108a within the first row and a neighboring graphical icon 108c within the second row.

This is beneficial for manufacturing purposes and simplifies printing of the graphical icons onto the first release liner 107a. Furthermore, the graphical icons within a first set of graphical icons are disposed in a communicative manner, such that the order of the steps can be followed easily.

The first release liner 107a and/or the edge release liners 107b-e may further comprise at least one printed texted word 114 or graphical branding, wherein the orientation of the printed texted word 114 or graphical branding indicates the orientation by which the medical dressing should be positioned onto the body part, i.e. the heel.

As used herein, the term “graphical branding” means a trademark, a logotype and/or a symbol intended to distinguish the product from other products.

The printed texted word 114 or graphical branding is preferably readable in the direction by which the dressing is to be positioned onto the body part (see 114 in FIG. 1a).

At least the first release liner 107a of the release liner assembly 107a-e may be transparent or white.

Typically, the edge release liner portions 107b-f are also transparent or white.

The transparent or white first release liner creates a visually distinct contrast between the graphical icons 108a-d, which are typically printed onto the first release liner, and the first release liner 107a.

Besides being difficult to apply, the dressing of the present disclosure is also difficult to package. After assembly, the dressing is typically packed in a sterile package, where it is stored until use. The large and complex shape of the dressing poses demands on the packaging procedure, which often requires the aid of an automated quality and process control device. The automated control device is configured to measure a number of packaging and product parameters. Such parameters include e.g. the pad length, the pad area, the pad position, potential skewness of the pad in the sterile package, proper sealing of the package etc.

The automated control device comprises a camera and illumination means. When the dressing has been packaged into the sterile package, the package is illuminated, and the different contrasts in the images are evaluated. One advantage of having a transparent or white release liner is that the white or transparent “color” does not “disturb” the illumination and control performed by the automated control device.

The first set of graphical icons 108a-d are typically disposed on the first release liner 107a such that at least 50%, preferably at least 70% of the graphical icons of the first set of graphical icons 108a-d overlap with the the pad 105.

As illustrated in FIG. 1a, the graphical icons 108a-d are preferably disposed in an area of the dressing where a pad is present. This is to enhance the contrast between the graphical icons and the first release liner 107a, being particularly advantageous if the first release liner is transparent. The underlying pad renders the graphical icons recognizable to a viewer.

Preferably, all of the graphical icons 108a-d are arranged to overlap with the the pad 105.

At least the first release liner 107a of the release liner assembly 107a-e may comprise a polymeric film selected from a polyethylene film, a polypropylene film and a polyurethane film.

A polymeric film allows for printing the graphical icons onto the first release liner. Furthermore, a polymeric film allows for welding, compressing and/or embossing the graphical icons onto the first release liner, if desired.

The first release liner 107a may be formed by the polymeric film or a laminate comprising the polymeric film. The thickness of the polymeric film is typically in the range of from 10 to 140 μm, e.g. from 20 to 100 μm. The first release liner preferably comprises a polyethylene or a polypropylene film having a thickness in the range of from 40 to 140 μm, e.g. from 70 to 100 μm.

The edge release liners 107b-e are typically formed from the same material as the first release liner. For example, the edge release liners 107b-e may comprise a polymeric film as described above.

Preferably, the at least one graphical icon 108a-d is a printed graphical icon formed by a colored ink.

Any colored ink may be used as long as it remains permanently adhered to the first release liner. Preferably, a flexographic ink is used. The ink may be water based or solvent based; i.e. the ink may comprise an organic solvent, such as an alcohol, ester etc. that can dissolve the pigment, resin and potentially other additives.

Preferably, the ink has a color that contrast with the color of the first release liner 107a. For example, the color of the ink may be grey, black, purple, pink, blue, green, red and any shades thereof.

In embodiments, the color of the ink is PMS 254 (Pantone Matching System).

In exemplary embodiments, at least the first release liner 107a of the release liner assembly 107a-e has a surface tension that is higher than the surface tension of the colored ink.

This allows for an improved adherence of the colored ink to the first release liner 107a. The surface tension of the first release liner may be increased by subjecting the first release liner to corona treatment.

A central area of the first release liner 107a is preferably void of printed graphical icons, and texted word(s) 114 or graphical branding(s), if present, wherein the central area circumvents the longitudinal (y) center line 101 and has a lateral extension of from 5 to 20%, preferably from 5 to 15% of the maximum lateral (x) extension of the first release liner 107a.

This is to avoid undesired disturbance of with the automated control system and to avoid the printed graphical icon(s) being falsely detected as a pad edge.

The width of the central area being void of printed graphical icon(s) is in FIG. 2, represented by d2. For example, the width, d2, may be from 0.5 to 4 cm, e.g. from 0.7 to 2 cm.

The colored ink preferably reflects light in the range of from 430 to 500 nm.

This is beneficial to avoid undesired disturbance with the automated control device. For example, a dark colored ink may be falsely detected as a pad edge, especially if the print is arranged next to the pad edges or its contour. With a colored ink reflecting light in the range of from 430 to 500 nm, the colored graphical icons are experienced as transparent by the automated control device. Accordingly, the risk of incorrect positioning or packaging in the sterile package is significantly reduced.

In the context of the present disclosure, the pad 105 preferably comprises a pressure-relieving material. Typically, the pad 105 is also absorbent.

The pad 105 may comprise one or more layers. For example, the pad may comprise a first absorbent layer, a liquid distributing layer and a second absorbent layer (not shown).

The liquid distributing layer may be arranged between the first and the second absorbent layers.

In exemplary embodiments, the first absorbent layer comprises an absorbent and hydrophilic foam, e.g. a polyurethane foam. The second absorbent layer may be a superabsorbent layer; i.e. a layer comprising a superabsorbent material. The superabsorbent layer has the capacity to absorb large amounts of wound exudate. The superabsorbent material may be in the form of particles, fibers, flakes or similar. For example, the superabsorbent material may comprise superabsorbent polymers (SAP) or superabsorbent fibers (SAF).

The liquid distributing layer may comprise any material having the ability to distribute the exudate in an efficient manner. For example, the liquid distributing layer may comprise a nonwoven material. A nonwoven imparts an appropriately balanced rigidity to the layer and to the dressing as such. It may also efficiently distribute and spread liquid absorbed by the first absorbent layer, e.g. the polyurethane foam, such that it can be evaporated through the backing layer over a large surface. For example, the nonwoven may comprise viscose, polyester or blends thereof.

The pad layers can be joined by adhesion or lamination, using e.g. pressure and heat.

A pad comprising a plurality of pad-forming layers as described hereinbefore prevents accumulation of body liquids close to the skin and improves the liquid handling of the dressing.

The backing layer 103 is the outermost layer of the dressing. The backing layer may e.g. comprise polyurethane, polyethylene or polypropylene. Typically, the backing layer comprises a polyurethane film. For example, the backing layer may comprise a polyurethane film having a thickness of from 10 μm to 50 μm, e.g. from 15 to 30 μm. The backing layer may be partly or fully attached to the pad, e.g. by means of an adhesive or by heat lamination

As used herein, the term “adhesive skin contact layer” means a layer configured to detachably adhere the dressing to a dermal surface. In other words, the adhesive skin contact layer is configured to contact the skin of a wearer.

Preferably, the adhesive skin contact layer comprises a silicone-based adhesive. A silicone-based adhesive is gentle to the skin and may be removed from the skin without causing any trauma. For example, the adhesive skin contact layer 104 may comprise a silicone gel. The silicone gel may e.g. be provided as a coating on the pad.

The adhesive skin contact layer 104 may comprise one or more sub-layers. As illustrated in FIG. 1b, the adhesive skin contact layer comprises a polymeric film 104a and a silicone gel layer 104b. The adhesive silicone gel layer 104b is arranged to contact the skin. Accordingly, the adhesive skin contact layer 104 may be a laminate comprising at least one polymeric film 104b and an adhesive silicone gel layer 104a.

The polymeric film simplifies the manufacturing process and provides stability and integrity to the silicone based skin contact layer. The polymeric film is preferably a breathable film and may comprise e.g. polyethylene, polyamide, polyester or polyurethane. Preferably, the polymeric film comprises polyurethane. The thickness of the polyurethane film may be from 15 to 100 μm, e.g. from 20 to 80 μm, preferably from 20 to 60 μm.

Examples of suitable silicone gels for use in the adhesive skin contact layer (103, 203) include the two component RTV systems, such as Q72218 (Dow Corning), and SilGel 612 (Wacker Chemie AG) mentioned herein, as well as NuSil silicone elastomers. In embodiments of the invention the adhesive may comprise a soft silicone gel having a softness (penetration) of from 8 to 22 mm, e.g. from 12 to 17 mm, as measured by a method based on ASTM D 937 and DIN 51580, the method being described in European Patent Application No 14194054.4. The thickness of the adhesive skin contact layer is typically at least 20 μm. The thickness of the adhesive skin contact layer may be from 50 to 200 μm.

According to another aspect, there is provided a method for manufacturing a medical dressing 100 comprising:
a) providing a medical dressing 100 having a longitudinal (y) extension and a lateral (x) extension; the medical dressing 100 being symmetric about a longitudinal (y) center line 101 and asymmetric about a lateral (x) center line 102; the dressing comprising a plurality of protruding portions 109a-d, wherein the dressing comprises a backing layer 103, an adhesive skin contact layer 104 and a pad 105 arranged between the backing layer 103 and the adhesive skin contact layer 104, wherein the backing layer 103 and the adhesive skin contact layer 104 extend beyond the periphery of the pad 105 to form a border portion 106 around the pad 105;
b) providing a release liner assembly 107a-e comprising a first release liner 107a and a plurality of edge release liners 107b-e;
c) printing at least one graphical icon 108a-d onto the first release liner 107a, wherein the graphical icon 108a-d indicates a correct application of the medical dressing 100 onto a body part, preferably the heel;
d) applying the plurality of edge release liners 107b-e onto the adhesive skin contact layer of at least parts of the protruding portions 109a-d of the medical dressing 100,
e) applying the first release liner 107a onto a central portion of the adhesive skin contact layer 104 such that the first release liner 107a covers from 50 to 90% of the surface area of the adhesive skin contact layer 104, and
f) trimming the first release liner 107a and the edge release liners 107b-e of the release liner assembly 107a-e such that a medical dressing comprising a release liner assembly 107a-e being co-extensive with the adhesive skin contact layer 104 is formed.

The medical dressing in step a) may be provided by means known to the skilled person. For example, the backing layer 103 may be adhesively attached to the pad 105. Alternatively, the backing layer 103 may be heat laminated onto the pad 105.

The adhesive skin contact layer 104 may be coated onto the pad 105 by techniques well known in the art. If the adhesive skin contact layer 104 comprises a polymeric film 104b and a silicone gel layer 104a, the adhesive skin contact layer is typically adhesively attached to the pad 105.

The graphical icon(s) 108a-d may be printed onto the first release liner 107a by any conventional printing technique known in the art, including, but not limited to a gravure printing, a flexographic printing, an offset printing, an ink jet printing and the like. Preferably, a flexographic printing technique is utilized.

The release liner assembly 107a-e may be applied by means known to the skilled person. Typically, the edge release liners 107b-e are first applied to the adhesive skin contact layer. The first, larger release liner 108b is then arranged onto a central portion of the adhesive skin contact layer 104 since this release liner portion is to be removed first from the dressing.

The first release liner 107a and the edge release liners 107b-e may be trimmed by any trimming or cutting technique known to the skilled person.

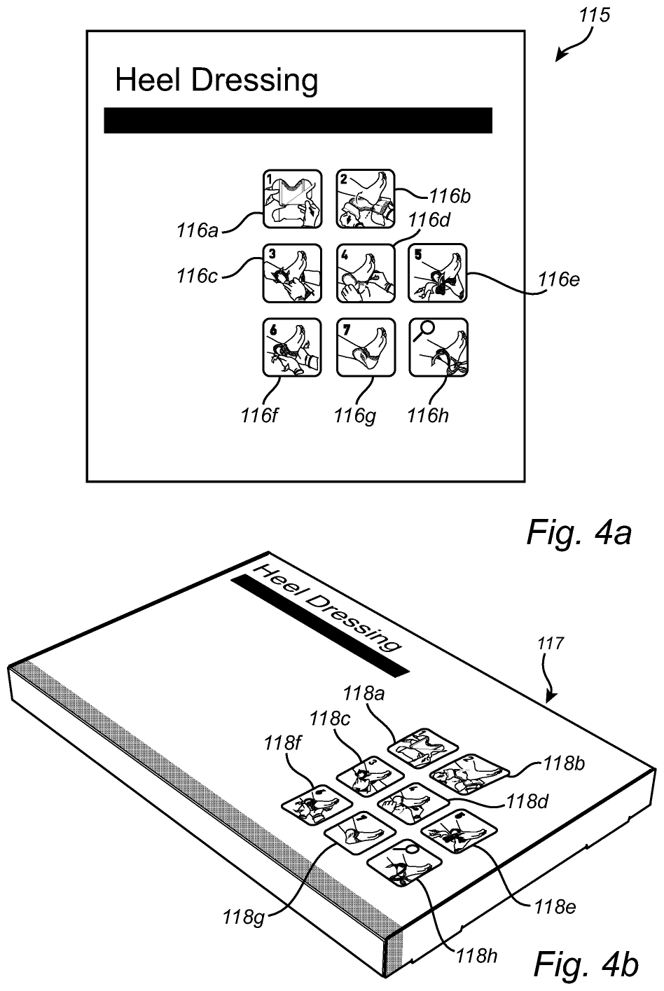

According to yet another aspect, illustrated in FIG. 4a, there is provided a sterile package 115 comprising the medical dressing 100 as described hereinbefore, wherein the sterile package comprises a second set of graphical icons 116a-h, wherein each graphical icon within the second set of graphical icons 116a-h comprises a pictorial representation of a step associated with the correct application of the medical dressing 100, and wherein at least one graphical icon within the first set of graphical icons 108a-d of the first release liner 107a corresponds to at least one graphical icon within the second set of graphical icons 116a-h.

The second set of graphical icons may comprise a more comprehensive set of graphical icons, comprising additional graphical icons with pictorial representations of additional steps in the application procedure.

In embodiments, at least two graphical icons, e.g. at least three or at least four graphical icons within the first set of graphical icons 108a-d of the first release liner 107a corresponds to at least two, e.g. at least three or four graphical icon within the second set of graphical icons 116a-h

According to yet another aspect, illustrated in FIG. 4b, there is provided, a multi-pack 117 comprising a plurality of sterile packages 115 as described hereinbefore, wherein the package 117 comprises a third set of graphical icons 118a-h, wherein each graphical icon within the third set of graphical icons 118a-h comprises a pictorial representation of a step associated with the correct application of the medical dressing 100, wherein at least one of the graphical icons within the third set of graphical icons 118a-h corresponds to at least one of the graphical icons within the first 108a-d and the second 116a-h sets of graphical icons.

Terms, definitions and embodiments of all aspects of the present disclosure apply mutatis mutandis to the other aspects of the present disclosure.

Even though the present disclosure has been described with reference to specific exemplifying embodiments thereof, many different alterations, modifications and the like will become apparent for those skilled in the art.

Variations to the disclosed embodiments can be understood and effected by the skilled addressee in practicing the present disclosure, from a study of the drawings, the disclosure, and the appended claims. Furthermore, in the claims, the word “comprising” does not exclude other elements or steps, and the indefinite article “a” or “an” does not exclude a plurality.

1. A medical dressing for application onto a body part, preferably the heel, wherein the dressing has a longitudinal (y) extension and a lateral (x) extension and is symmetric about a longitudinal (y) center line and asymmetric about a lateral (x) center line; said medical dressing comprising a backing layer, an adhesive skin contact layer and a pad arranged between said backing layer and said adhesive skin contact layer, wherein said dressing comprises a release liner assembly being co-extensive with said adhesive skin contact layer, wherein said release liner assembly comprises a centrally disposed first release liner covering from 50 to 90% of the surface area of said adhesive skin contact layer, and a plurality of edge release liners, characterized in that said first release liner comprises at least one graphical icon indicating a correct application of said medical dressing onto said body part.

2. The medical dressing according to claim 1, wherein said backing layer (103) and said adhesive skin contact layer extend beyond the periphery of said pad to form a border portion around said pad.

3. The medical dressing according to claim 1, wherein said medical dressing comprises a plurality of protruding portions, and wherein said edge release liners are arranged to cover at least parts of said protruding portions.

4. The medical dressing according to claim 1, wherein said at least one graphical icon is disposed in an area of said first release liner that overlaps with said pad.

5. The medical dressing according to claim 1, wherein said first release liner is symmetric about said longitudinal (y) center line and has a longitudinal extension corresponding to the longitudinal extension of said adhesive skin contact layer in the area where the first release liner is arranged, and a maximum lateral (x) extension corresponding to from 50-90% of the maximum lateral (x) extension of said adhesive skin contact layer.

6. The medical dressing according to claim 1, wherein said first release liner is configured to be removed first from said adhesive skin contact layer.

7. The medical dressing according to claim 1, wherein said first release liner comprises at least a first set of graphical icons, wherein each graphical icon within said first set of graphical icons comprises a pictorial representation of a step associated with the correct application of said medical dressing onto said body part, preferably wherein said body part is the heel, preferably wherein said step associated with the correct application of said medical dressing is selected from: a correct removal and/or order of removal of said release liner assembly (107a-e), a correct positioning of said medical dressing onto the heel, a correct application of said dressing onto the heel, and a medical dressing applied correctly onto the heel.

8. (canceled)

9. The medical dressing according to claim 7, wherein at least one graphical icon within said first set of graphical icons comprises a pictorial representation indicating the correct positioning of said medical dressing onto the heel, and wherein said pictorial representation comprises a guiding line indicating the correct positioning of the medical dressing onto the heel.

10. The medical dressing according to claim 7, wherein said first set of graphical icons comprises at least four graphical icons, and wherein the graphical icons of said first set of graphical icons are arranged in at least a first row and a second row on said first release liner, preferably wherein the distance, d1, between a first graphical icon of said first row and a neighboring graphical icon within said first row is the same as the distance, d2, between the first graphical icon within said first row and a neighboring graphical icon within said second row.

11. (canceled)

12. The medical dressing according to claim 1, wherein said first release liner and/or said edge release liners further comprise(s) at least a printed texted word and/or graphical branding, wherein the orientation of said printed texted word and/or graphical branding indicates the orientation by which the dressing should be positioned onto said body part, preferably wherein said at least one printed texted word (114) and/or graphical branding is readable in the direction by which the dressing is to be positioned onto said body part.

13. (canceled)

14. The medical dressing according to claim 1, wherein at least said first release liner of said release liner assembly is transparent or white.

15. The medical dressing according to claim 7, wherein said first set of graphical icons are disposed on said first release liner (107a) such that at least 50% of the graphical icons of said first set of graphical icons overlap with said pad.

16. The medical dressing according to claim 1, wherein at least said first release liner of said release liner assembly comprises a polymeric film selected from a polyethylene film, a polypropylene film and a polyurethane film.

17. The medical dressing according to claim 1, wherein said at least one graphical icon is a printed graphical icon formed by a colored ink.

18. The medical dressing according to claim 17, wherein at least said first release liner of said release liner assembly has a surface tension that is higher than the surface tension of said colored ink.

19. The medical dressing according to claim 17, wherein a central area of said first release liner is void of printed graphical icon(s) and texted word(s) or graphical brandings, if present, wherein said central area is an area circumventing said longitudinal center line and having a lateral extension of from 5 to 20% of the maximum lateral extension of said first release liner.

20. The medical dressing according to claim 17, wherein said colored ink reflects light in the range of from 430 to 500 nm.

21.

22. A sterile package comprising the medical dressing according to claim 7, wherein said sterile package comprises a second set of graphical icons, wherein each graphical icon within said second set of graphical icons comprises a pictorial representation of a step associated with the correct application of said medical dressing, and wherein at least one graphical icon within said first set of graphical icons of said first release liner corresponds to at least one graphical icon within said second set of graphical icons.

23. A multi-pack comprising a plurality of sterile packages according to claim 22, wherein said multi-pack comprises a third set of graphical icons, wherein each graphical icon within said third set of graphical icons comprises a pictorial representation of a step associated with the correct application of said medical dressing, wherein at least one of the graphical icons within said third set of graphical icons corresponds to at least one of the graphical icons within said first and said second sets of graphical icons.
